(19) United States
(12) Patent Application Publication
(10) Pub. No.: US 2023/0338704 A1
(43) Pub. Date: Oct. 26, 2023

(54) MEDICAL DEVICE RELEASE SYSTEM

(71) Applicant: BOSTON SCIENTIFIC SCIMED, INC., MAPLE GROVE, MN (US)

(72) Inventors: Ryan P Foote, Minneapolis, MN (US); Eric Douglas Nygaard, Eden Prairie, MN (US); Nicholas Lee Tassoni, Andover, MN (US)

(73) Assignee: BOSTON SCIENTIFIC SCIMED, INC., MAPLE GROVE, MN (US)

(21) Appl. No.: 18/137,675

(22) Filed: Apr. 21, 2023

Related U.S. Application Data
(60) Provisional application No. 63/333,658, filed on Apr. 22, 2022.

(51) Int. Cl. A61M 25/00; A61M 25/01

(52) U.S. Cl. CPC A61M 25/0074; A61M 25/0147; A61M 2025/015; A61M 2205/0216

(57) ABSTRACT

A medical device system may include an elongate shaft having a lumen extending from a proximal end of the elongate shaft to a distal end of the elongate shaft, a medical device disposed proximate the distal end of the elongate shaft, a release wire disposed within the lumen of the elongate shaft, wherein the release wire releasably secures the medical device to the distal end of the elongate shaft, a securement member fixedly attached to the proximal end of the elongate shaft and to a proximal end of the release wire, and a microcatheter configured to deliver the medical device to a treatment site, the elongate shaft and the medical device being slidably disposed within a lumen of the microcatheter. A proximal portion of the securement member may be configured to disengage from a distal portion of the securement member.

### CROSS-REFERENCE TO RELATED APPLICATIONS

This application claims the benefit of priority of U.S. Provisional Application No. 63/333,658 filed Apr. 22, 2022, the entire disclosure of which is hereby incorporated by reference.

### TECHNICAL FIELD

The present disclosure pertains to medical devices and methods for manufacturing and/or using medical devices. More particularly, the present disclosure pertains to configurations of a system for releasing medical implants.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

The disclosure pertains to configurations of a system for releasing medical implants. An example may be found in a medical device system. The medical device system includes a medical device and an elongate shaft having a lumen extending from a proximal end of the elongate shaft to a distal end of the elongate shaft, the medical device securable to the distal end of the elongate shaft. A release mechanism is moveable between a locked position in which the medical device is secured to the distal end of the elongate shaft and an unlocked position in which the medical device is free to move relative to the distal end of the elongate shaft. A release wire is disposed within the lumen of the elongate shaft and is extendable through the release mechanism. A securement member includes a release point formed within the securement member, a proximal portion extending proximally from the release point and a distal portion extending distally from the release point. The release point of the securement member is configured to allow a user to grasp the proximal portion of the securement member and the distal portion of the securement member on either side of the release point and to snap the securement member into two pieces at the release point in a single motion.

Alternatively or additionally, the release wire may extend through the securement member, with the release wire fixed relative to the proximal portion of the securement member such that once the securement member has been snapped at the release point, the proximal portion of the securement member is movable in a proximal direction in order to urge the release wire in a proximal direction.

Alternatively or additionally, the release wire may form a frictional fit with the proximal portion of the securement member.

Alternatively or additionally, the proximal portion of the securement member may be crimped down onto the release wire.

Alternatively or additionally, the release wire may be welded, soldered or adhesively secured to the proximal portion of the securement member.

Alternatively or additionally, the release wire may be free to move relative to the distal portion of the securement member.

Alternatively or additionally, the release mechanism may include a first release mechanism portion that is fixedly attached to the distal end of the elongate shaft, and second release mechanism portion that is fixedly attached to the medical device.

Alternatively or additionally, the securement member may include a tubular structure made of a nickel-titanium alloy.

Alternatively or additionally, the tubular structure may include an inner diameter (ID) that is in a range of 0.001 to 0.0049 inches, and an outer diameter (OD) that is in a range of 0.002 to 0.0050 inches.

Alternatively or additionally, the release point may include a plurality of slots extending radially around the tubular structure at the release point.

Alternatively or additionally, the plurality of slots may be radially aligned with one another, forming a plurality of beams extending radially between adjacent slots.

Alternatively or additionally, the release point may include a total of four radially aligned slots, with each of the slots having a length of 0.010 inches.

Alternatively or additionally, the proximal portion of the securement member may be fixedly attached to the proximal end of the release wire and the distal portion of the securement member may be fixedly attached to the proximal end of the elongate shaft.

Alternatively or additionally, an outer surface of the distal portion of the securement member may be fixedly attached to an inner surface of the elongate shaft.

Alternatively or additionally, the proximal portion of the securement member may be integrally formed with the distal portion of the securement member.

Another example may be found in a medical device system. The medical device system includes a medical device and an elongate shaft having a lumen extending from a proximal end of the elongate shaft to a distal end of the elongate shaft, the medical device securable to the distal end of the elongate shaft. A release mechanism is moveable between a locked position in which the medical device is secured to the distal end of the elongate shaft and an unlocked position in which the medical device is free to move relative to the distal end of the elongate shaft. A release wire is disposed within the lumen of the elongate shaft and is extendable through the release mechanism. A tubular super-elastic nitinol securement member includes a release point formed within the securement member, a proximal portion extending proximally from the release point and a distal portion extending distally from the release point.

The release point includes a total of four circumferentially-arranged slots formed within the tubular super-elastic nitinol securement member, the release point being configured to allow a user to grasp the proximal portion of the securement member and the distal portion of the securement member on either side of the release point and to snap the securement member into two pieces at the release point in a single motion

Alternatively or additionally, the securement member may include an inner diameter (ID) that is in a range of 0.001 to 0.0049 inches, and an outer diameter (OD) that is in a range of 0.002 to 0.0050 inches, and the release point may include a plurality of slots extending radially around the tubular structure.

Another example may be found in a method of delivering a medical device to a treatment site. The method includes inserting a microcatheter into a patient's anatomy and guiding a distal end of the microcatheter to a location adjacent the treatment site, and inserting a medical device disposed at a distal end of an elongate shaft into a proximal end of a lumen disposed within the microcatheter. The medical device is releasably attached to the distal end of the elongate shaft by a pull wire extending through a lumen within the elongate shaft, and wherein a securement member extends proximally from the elongate shaft, the securement member being fixedly attached to the elongate shaft and the pull wire, the securement member including a release point. The medical device is advanced through the microcatheter to the treatment site. The securement member is grasped on either side of the release point and the securement member is snapped in two pieces at the release point with a single motion, with the pull wire fixedly secured to a proximal portion of the securement member. The pull wire is translated relative to the elongate shaft by moving the proximal portion of the securement member proximally, thereby releasing the medical device from the elongate shaft.

Alternatively or additionally, the proximal portion of the securement member may be fixedly attached to the pull wire and the distal portion of the securement member is fixedly attached to the elongate shaft.

Alternatively or additionally, a first portion of a release mechanism may be attached to the distal end of the elongate shaft and a second portion of the release mechanism may be attached to a proximal end of the medical device.

Alternatively or additionally, the pull wire may be slidably disposed within the distal portion of the securement member, the elongate shaft, the first portion of the release mechanism, and the second portion of the release mechanism.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative medical device system;

FIG. 2 is a partial cut-away view of an illustrative medical device system;

FIG. 3 is a partial cut-away view of a portion of an illustrative medical device system;

FIG. 3A is a partial cut-away view of a portion of an illustrative medical device system;

FIG. 3B is a partial cut-away view of a portion of an illustrative medical device system;

FIG. 4 is a partial cut-away view of a portion of an illustrative medical device system;

FIGS. 5-6 are partial cut-away views illustrating actuation of a portion of an illustrative medical device system;

FIG. 7 shows an example release mechanism of an illustrative medical device system;

FIG. 8 shows a portion of an illustrative securement member of an illustrative medical device system;

FIG. 9 is a cross-sectional view taken along line 9-9 of FIG. 8;

FIG. 10 shows a portion of an example securement member of an illustrative medical device system; and

FIG. 11 and FIG. 12 together provide an example of snapping an example securement member.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

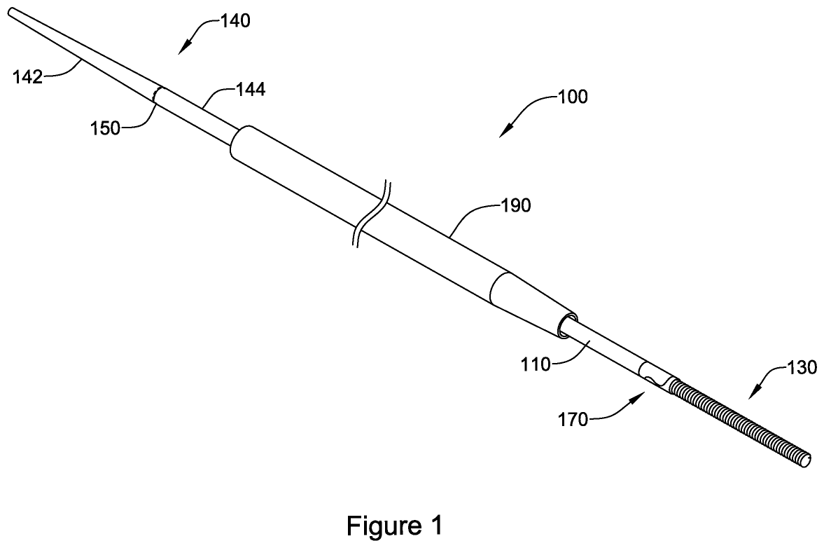

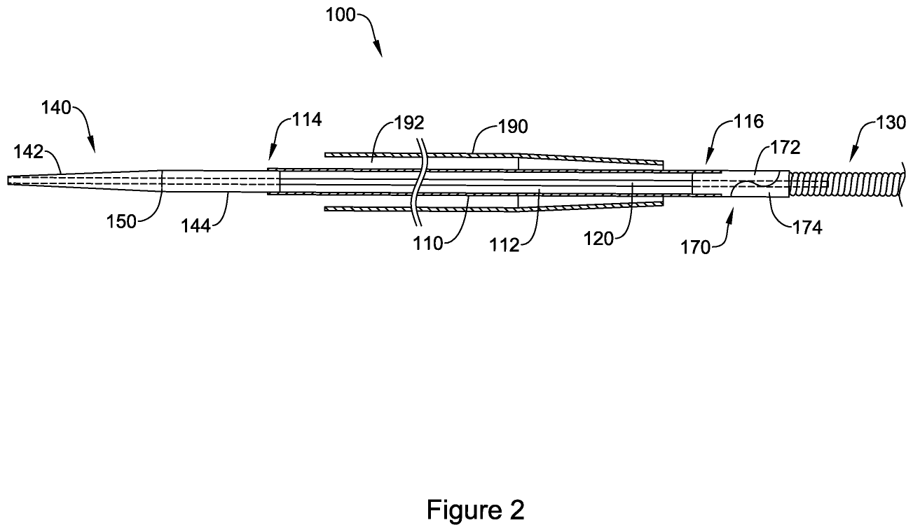

FIGS. 1 and 2 illustrate features of an example medical device system 100. The medical device system 100 may include an elongate shaft 110 having a lumen 112 extending from a proximal end 114 of the elongate shaft 110 to a distal end 116 of the elongate shaft 110. In some embodiments, the elongate shaft 110 may be a catheter, a hypotube, or other similar tubular structure. Some suitable but non-limiting materials for the elongate shaft 110, for example metallic materials, polymer materials, composite materials, etc., are described below.

The medical device system 100 may include a release wire 120 slidably disposed within the lumen 112 of the elongate shaft 110. A medical device 130 may be disposed proximate the distal end 116 of the elongate shaft 110. The release wire 120 may be configured to releasably attach the medical device 130 to the distal end 116 of the elongate shaft 110. For simplicity, the medical device 130 is illustrated herein as a shape memory embolic coil, such as those used to treat aneurysms for example, but other suitable medical devices transported, delivered, used, released etc. in a similar manner are also contemplated, including but not limited to stents, embolic filters, replacement heart valves, occlusion devices, and/or other medical implants, etc. In some embodiments, the release wire 120 may be alternately and/or interchangeably referred to as a pull wire, an actuation wire, and/or a locking wire. The release wire 120 may generally be a solid wire or shaft, but may also be tubular in some embodiments. Some suitable but non-limiting materials for the release wire 120, for example metallic materials, polymer materials, composite materials, etc., are described below.

In some embodiments, the medical device system 100 may include a microcatheter 190 sized and configured to deliver the medical device 130 to a treatment site. The elongate shaft 110 and the medical device 130 may be slidably disposed within a lumen 192 of the microcatheter 190. In some embodiments, the microcatheter 190 may facilitate percutaneous delivery of the medical device 130 to the treatment site. Some suitable but non-limiting materials for the microcatheter 190, for example metallic materials, polymer materials, composite materials, etc., are described below.

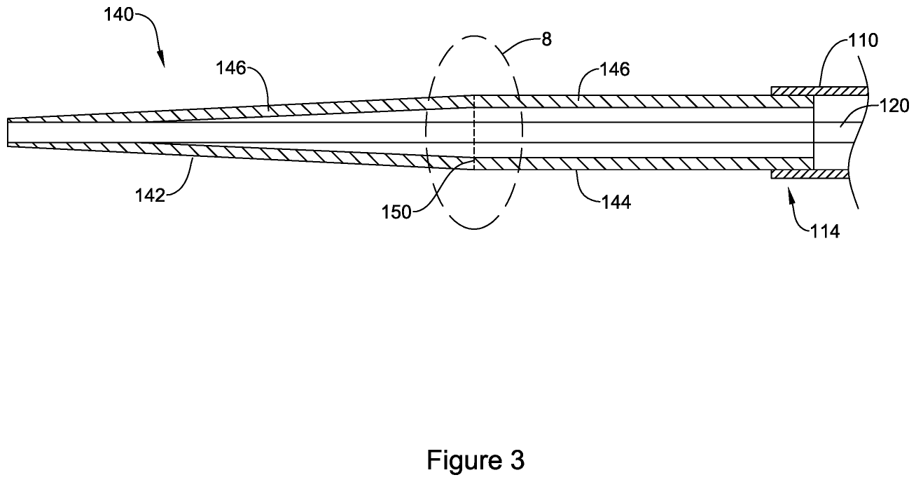

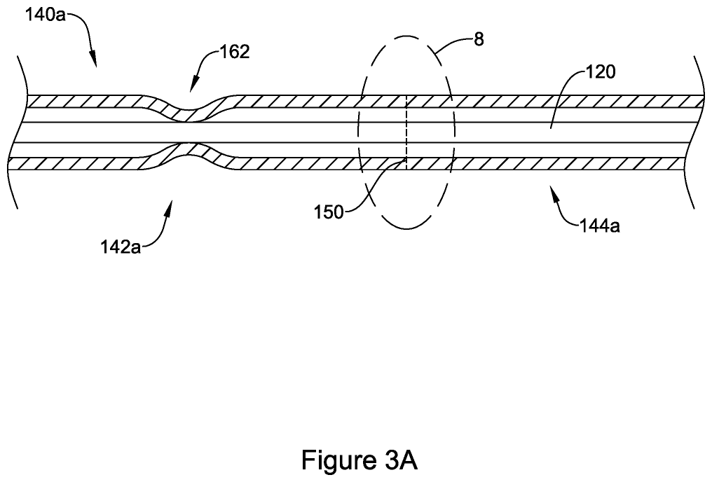

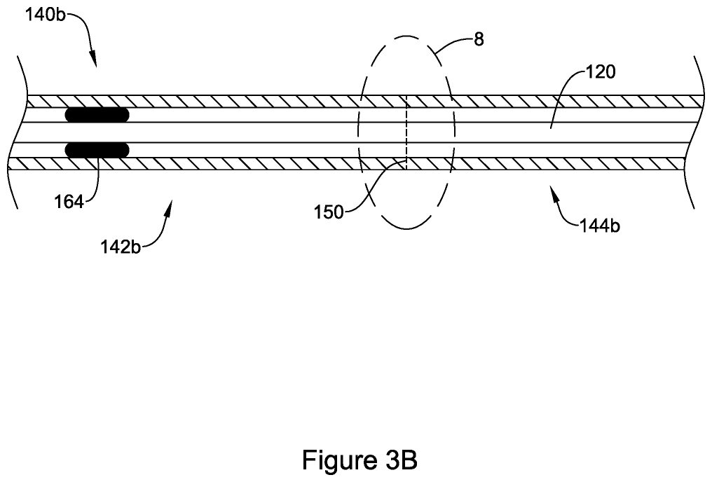

As seen for example in FIG. 3, the medical device system 100 may include a securement member 140 fixedly attached to and/or extending proximally from the proximal end 114 of the elongate shaft 100, and fixedly attached to a proximal end of the release wire 120. The securement member 140 may include a proximal portion 142, a distal portion 144, and a wall 146 extending from a proximal end of the securement member 140 to a distal end of the securement member 140. In at least some embodiments, the proximal portion 142 of the securement member 140 may be integrally formed with the distal portion 144 of the securement member 140 as a single unitary structure. Some suitable but non-limiting materials for the securement member 140, for example metallic materials, polymer materials, composite materials, etc., are described below. In some cases, the securement member 140 is made of a nickel-titanium alloy such as nitinol. In some instances, the securement member 140 is made of super-elastic nitinol.

The proximal portion 142 of the securement member 140 may be fixedly attached to the proximal end of the release wire 120. The distal portion 144 of the securement member 140 may be fixedly attached to the proximal end 114 of the elongate shaft 110. In at least some embodiments, an outer surface of the distal portion 144 of the securement member 140 may be fixedly attached to an inner surface of the elongate shaft 110 (e.g., a surface defining the lumen 112). Alternatively, in some embodiments, an inner surface of the distal portion 144 of the securement member 140 may be fixedly attached to an outer surface of the elongate shaft 110.

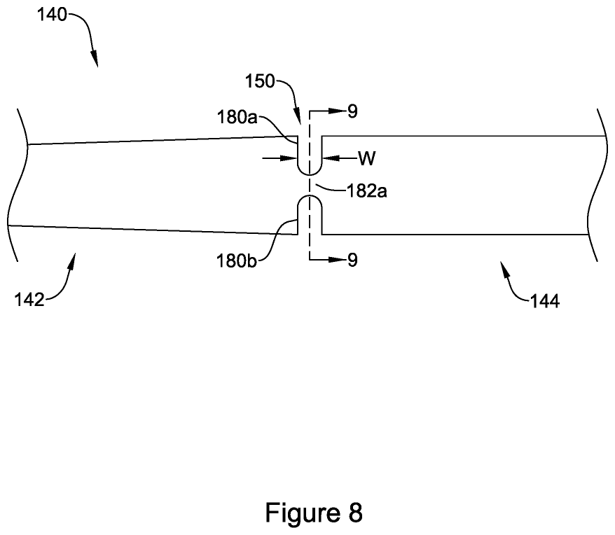

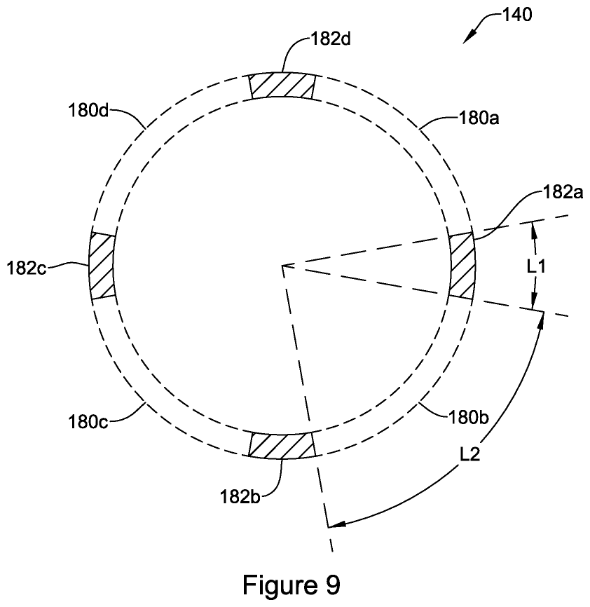

As will be discussed in greater detail with respect to FIGS. 8 and 9, the securement member 140 may include a release point 150 that is configured to allow the securement member 140 to remain intact while advancing the medical device system 100 through the anatomy. The release point 150 is configured to allow an operator of the medical device system 100 to easily snap the securement member 140 into two pieces, in a single motion, in order to release the medical device 130. In some embodiments, the proximal portion 142 of the securement member 140 may be releasably secured to and/or configured to disengage from the distal portion 144 of the securement member 140 at the release point 150 formed in the wall 146 of the securement member 140.

In some instances, the securement member 140 may be formed of a nickel-titanium alloy such as nitinol. In some instances, the securement member 140 may be formed of super-elastic nitinol. Other materials such as stainless steel, were found to require multiple motions to fatigue the material before the material would snap. Having the securement member 140 formed of super-elastic nitinol, in combination with the slots formed within the securement member 140 at the release point 150 (as will be discussed) provides the securement member 140 with an ability to easily be snapped in half with a single motion. In some instances, using super-elastic nitinol for the securement member 140 helps to prevent undesirable kinking.

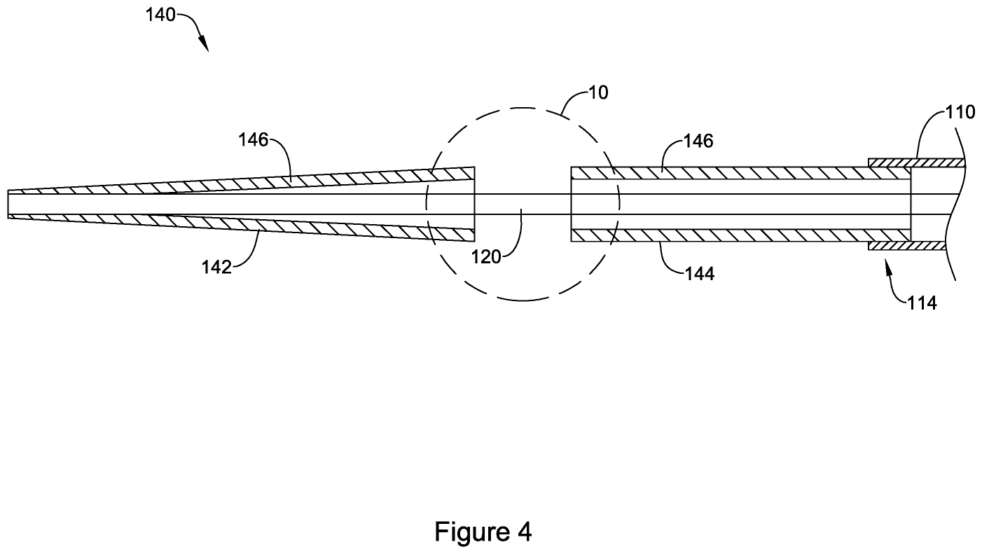

In at least some embodiments, the securement member 140 may prevent axial translation of the release wire 120 relative to the elongate shaft 110 and/or the medical device 130 prior to snapping the securement member 140 into two pieces at the release point 150, thereby disengaging the proximal portion 142 of the securement member 140 from the distal portion 144 of the securement member 140. Disengaging the proximal portion 142 of the securement member 140 from the distal portion 144 of the securement member 140 may permit the release wire 120 to axially translate relative to the distal portion 144 of the securement member 140 and/or the elongate shaft 110. In other words, the wall 146 of the distal portion 144 of the securement member 140 may define a lumen, as seen in FIGS. 3 and 4 for example, wherein the release wire 120 is slidably disposed within the lumen of the distal portion 144 of the securement member 140.

In some instances, the release wire 120 is constrained from axial movement relative to the proximal portion 142 of the securement member 140 but is not otherwise constrained from axial movement relative to the distal portion 144 of the securement member 140. As seen for example in FIG. 3, the inner diameter of the proximal portion 142 of the securement member 140 may taper to a dimension equal or substantially equal to an outer diameter of the release wire 120. The release wire 120 may have a frictional fit with the proximal portion 142 of the securement member 140 such that when the securement member 140 is snapped into two pieces at the release point 150, the proximal portion 142 of the securement member 140 remains attached to the release wire 120. As a result, the operator is able to cause the release wire 120 to translate axially by moving the proximal portion 142 of the securement member 140 axially, without any additional handle. Put another way, the proximal portion 142 of the securement member 140, once the securement member 140 has been snapped into two pieces, functions as a handle for grasping and translating the release wire 120. The inner diameter of the distal portion 144 of the securement member 140 is greater than the outer diameter of the release wire 120, and hence the distal portion 144 of the securement member 140 does not constrain the release wire 120 from axial movement relative to the distal portion 144 of the securement member 140.

Upon disengagement of the proximal portion 142 of the securement member 140 from the distal portion 144 of the securement member 140, as seen in FIG. 4, axial translation of the proximal portion 142 relative to the distal portion 144 of the securement member 140 and/or the elongate shaft 110 may translate the release wire 120 relative to the elongate shaft 110 and/or the distal portion 144 of the securement member 140 to release the medical device 130 from the distal end 116 of the elongate shaft 110, as will be explained in more detail herein.

In some instances, as shown in FIG. 3, the proximal portion 142 of the securement member 140 may taper in diameter moving proximally from the release point 150. In some instances, the proximal portion 142 may not taper in this manner. FIG. 3A shows a securement member 140a that may be used in place of the securement member 140 in the medical device system 100, for example. The securement member 140a includes a proximal portion 142a and a distal portion 144a, with the release point 150 disposed between the proximal portion 142a of the securement member 140a and the distal portion 144a of the securement member 140a. The release wire 120 is constrained from axial movement relative to the proximal portion 142a of the securement member 140a by one or more crimps 162 that are formed in the proximal portion 142a. While a single crimp 162 is shown, extending circumferentially around the proximal portion 142a of the securement member 140a, it will be appreciated that in some instances there may be two, three or more crimps 162 extending circumferentially around the proximal portion 142a of the securement member 140a. The securement member 140a is configured to easily snap, in a single motion applied by the operator, at the release point 150.

FIG. 3B shows a securement member 140b that may be used in place of the securement member 140 in the medical device system 100, for example. The securement member 140b includes a proximal portion 142b and a distal portion 144b, with the release point 150 disposed between the proximal portion 142b of the securement member 140b and the distal portion 144b of the securement member 140b. The release wire 120 is constrained from axial movement relative to the proximal portion 142b of the securement member 140b by one or more fixation elements 164 that are formed in the proximal portion 142b. The one or more fixation elements 164 may be welds, for example. The one or more fixation elements 164 may be solder. In some instances, the one or more fixation elements 164 may be adhesive. While a single fixation element 164 is shown, extending circumferentially around the proximal portion 142b of the securement member 140b, it will be appreciated that in some instances there may be two, three or more fixation elements 164 extending circumferentially around the proximal portion 142b of the securement member 140b. The securement member 140a is configured to easily snap, in a single motion applied by the operator, at the release point 150. It will be appreciated that other systems and techniques for securing the release wire 120 against axial movement relative to the proximal portion 142b of the securement member 140b are contemplated.

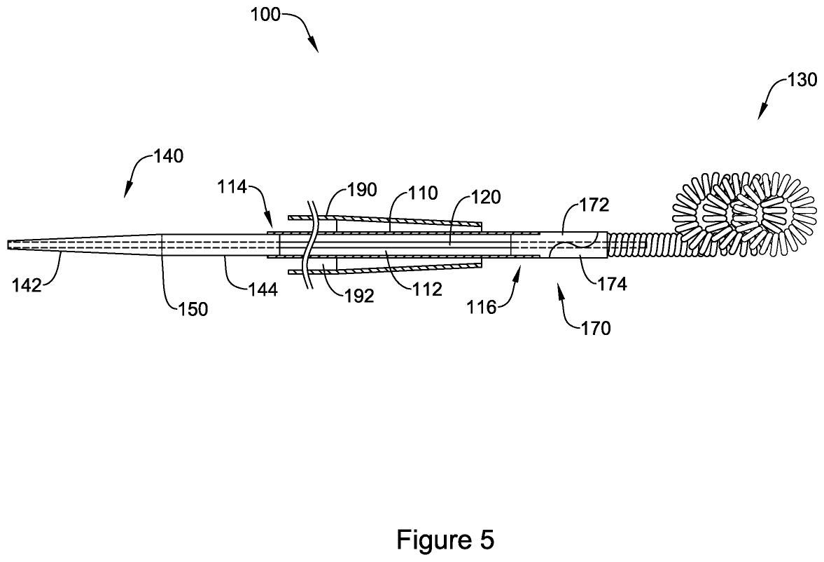

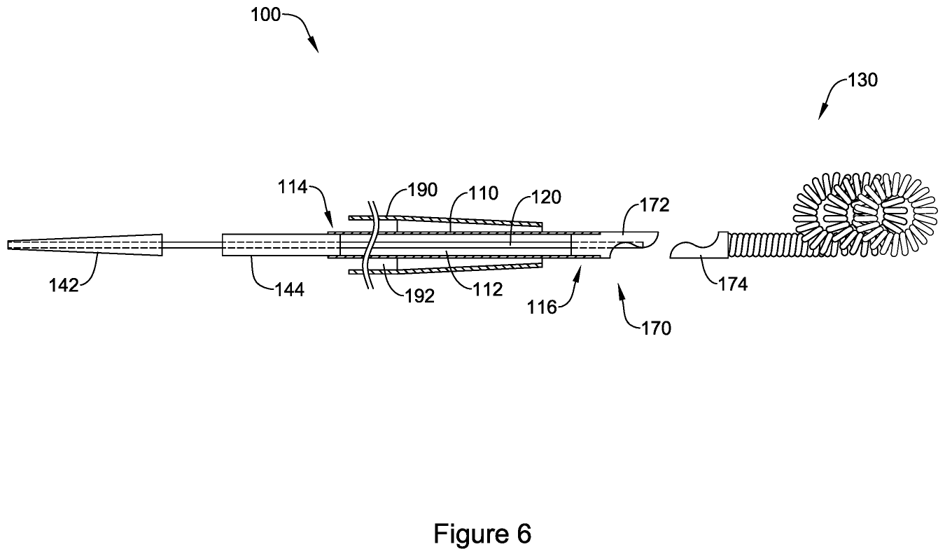

FIGS. 5 and 6 generally illustrate the medical device 130 being released from the elongate shaft 110, such as at a treatment site, for example. In use, the microcatheter 190 of the medical device system 100 may be inserted into a patient's anatomy and a distal end guided and/or advanced to a location adjacent a treatment site. The medical device 130 disposed at the distal end 116 of the elongate shaft 110 may be inserted into a proximal end of the lumen 192 disposed within the microcatheter 190 and advanced through the microcatheter 190 to the treatment site. In some embodiments, the medical device 130 may be disposed within the lumen 192 of the microcatheter 190 proximate to the distal end 116 of the elongate shaft 110. In some embodiments, the medical device 130 may be disposed within the lumen 192 of the microcatheter 190 proximate to the distal end 116 of the elongate shaft 110 prior to use and/or prior to inserting the microcatheter 190 into the patient's anatomy. Deployment and/or release of the medical device 130 may be performed selectively depending upon the type of medical device and/or the desired treatment process or method. When ready to deploy the medical device 130, the elongate shaft 110 may be advanced and/or translated distally relative to the microcatheter 190 until the medical device 130 is exposed and/or disposed distal of the microcatheter 190.

A release mechanism 170 may releasably attach the medical device 130 to the distal end 116 of the elongate shaft 110. In some embodiments, the elongate shaft 110 may include a first portion 172 of the release mechanism 170 fixedly attached to the distal end 116 of the elongate shaft 110 and the medical device 130 may include a second portion 174 of the release mechanism 170 fixedly attached to a proximal end of the medical device 130. A distal end of the release wire 120 may slidably engage with the first portion 172 of the release mechanism 170 and the second portion 174 of the release mechanism 170, as seen in FIG. 5. The release wire 120 interlocks the first portion 172 of the release mechanism 170 with the second portion 174 of the release mechanism 170 when the proximal portion 142 of the securement member 140 is engaged with the distal portion 144 of the securement member 140, thereby preventing the first portion 172 of the release mechanism 170 from moving relative to the second portion 174 of the release mechanism 170.

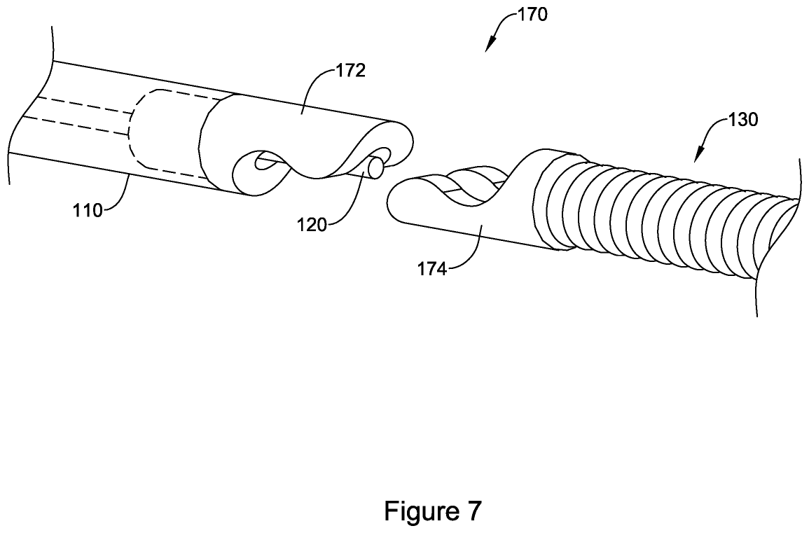

For example, when the proximal portion 142 of the securement member 140 has been separated from the distal portion 144 of the securement member 140 by snapping the securement member 140 into two pieces at the release point 150, as seen in FIG. 6, the release wire 120 may be translated in a proximal direction relative to the elongate shaft 110 to release the second portion 174 of the release mechanism 170 and/or the medical device 130 from the first portion 172 of the release mechanism 170 and/or the elongate shaft 110, as seen in more detail in FIG. 7. In at least some embodiments, the release wire 120 may be slidably disposed within the distal portion 144 of the securement member 140, the elongate shaft 110, the first portion 172 of the release mechanism 170, and the second portion 174 of the release mechanism 170. Some suitable but non-limiting materials for the release mechanism 170, for example metallic materials, polymer materials, composite materials, etc., are described below.

Referring back to FIGS. 5 and 6, the elongate shaft 110 may have sufficient length that the proximal end 114 of the elongate shaft 110 and/or the securement member 140 remain proximal of (e.g., extend proximally from) the microcatheter 190 when the medical device 130 is disposed distal of the microcatheter 190. In use, the elongate shaft 110 may have sufficient length to reach from the treatment site to a position outside of the patient where the medical device system 100 may be manipulated by an operator (e.g., clinician, physician, user, etc.).

In some instances, the proximal portion 142 of the securement member 140 may be configured to disengage from the distal portion 144 of the securement member 140 at a location proximal of a proximal end of the microcatheter 190 when the medical device 130 is disposed distal of the microcatheter 130. In at least some embodiments, the proximal portion 142 of the securement member 140 may be disengaged from the distal portion 144 of the securement member 140 by snapping the securement member 140 at the release point 150. In some embodiments, disengaging the proximal portion 142 of the securement member 140 from the distal portion 144 of the securement member 140 may include moving the proximal portion 142 of the securement member 140 relative to the distal portion 144 of the securement member 140 to separate the proximal portion 142 of the securement member 140 from the distal portion 144 of the securement member 140 after snapping the securement member 140 into two pieces at the release point 150.

FIG. 8 illustrates the securement member 140 including the release point 150 that is formed within the wall 146 of the securement member 140. FIG. 9 is a cross-sectional view taken along the line 9-9 of FIG. 8. As shown, the release point 150 includes a number of circumferentially-extending slots 180, individually labeled as 180a, 180b, 180c and 180d. The circumferentially-extending slots 180 are separated by beams 182, individually labeled as 182a, 182b, 182c and 182d. While a total of four circumferentially-extending slots 180 and a total of four beams 182 are shown, it will be appreciated that in some cases, there may be only three circumferentially-extending slots 180 and only three intervening beams 182. In some cases, there may be five or more circumferentially-extending slots 180 and five or more intervening beams 182.

In some cases, each of the circumferentially-extending slots 180 have the same width, indicated by a width “W”. In some cases, the width “W” may vary depending on other dimensions of the securement member 140. For example, as the inner diameter (ID) and the outer diameter (OD) of the securement member 140, and hence the resulting wall thickness therebetween, varies, the width “W” varies. In some cases, the securement member 140 may have an ID that ranges from 0.01 to 0.049 inches and an OD that ranges from 0.002 to 0.050 inches. In some cases, the circumferentially-extending slots 180 may have a width “W” that is in a range of 0.0015 inches to 0.0035 inches. The circumferentially-extending slots 180 may have a width “W” of 0.0025 inches, for example.

In an example, the securement member 140 has an ID of 0.008 inches and an OD of 0.018 inches. A beam length of 0.0050 inches corresponds to a slot length of 0.0091 inches. A beam length of 0.0055 inches corresponds to a slot length of 0.0086 inches. A beam length of 0.0060 inches corresponds to a slot length of 0.0081 inches. A beam length of 0.0065 inches corresponds to a slot length of 0.0076. A beam length of 0.0070 inches corresponds to a slot length of 0.0071 inches. A beam length of 0.0075 inches corresponds to a slot length of 0.0066 inches. Increasing the beam length (and decreasing the corresponding slot length) strengthens the securement member 140, but makes the securement member 140 harder to snap. Shortening the beam length (and increasing the corresponding slot length) makes the securement member 140 easier to snap, but may make the securement member 140 more prone to premature breakage.

While the circumferentially-extending slots 180 are shown as having a rounded terminus, in some cases, the circumferentially-extending slots 180 may instead have a rectilinear terminus, a triangular terminus, or any other of a variety of terminus shapes. The circumferentially-extending slots 180 may be laser-cut, for example, although saw cutting may also be performed to create or modify the circumferentially-extending slots 180.

As seen for example in FIG. 9, each of the beams 182 have an arc length indicated by “L₁” and each of the circumferentially-extending slots 180 have an arc length indicated by “L₂”. As shown, the arc length of “L₁” is shorter than the arc length of “L₂”. In some cases, the arc length “L₁” may range from 0.005 inches to 0.010 inches and the arc length of “L₂” may range from 0.010 inches to 0.020 inches, for example. For a securement member 140 having an ID of 0.008 inches and an OD of 0.018 inches, each of the circumferentially-extending slots 180 may have a slot length of 0.010 inches. For a smaller diameter securement member 140 having an OD of 0.002 inches, the slot length is 0.001 inches. For a securement member 140 having an OD of 0.050 inches, the slot length may be 0.025 inches.

It will be appreciated that the arc lengths “L₁” and “L₂” are measured along the curved outer surface of the securement member 140. In some cases, the four beams 182 may, in total, consume about 30 percent of the 360-degree circumference of the securement member 140 at the release point 150, and the four slots 180 may, in total, consume about 70 percent of the 360 degree circumference of the securement member 140 at the release point 150.

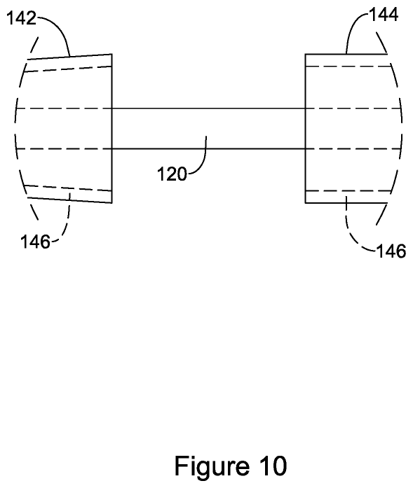

FIG. 10 (detail “10” from FIG. 4) illustrates the securement member 140 after disengaging the proximal portion 142 of the securement member 140 from the distal portion 144 of the securement member 140 at the perforation 150 and/or the frangible link 160 and subsequent separation of the proximal portion 142 of the securement member 140 from the distal portion 144 of the securement member 140. Proximal translation of the proximal portion 142 of the securement member 140 relative to the distal portion 144 of the securement member 140 and/or the elongate shaft 110 results in the release wire 120 translating and/or sliding within and/or relative to the elongate shaft 110 and the distal portion 144 of the securement member 140.

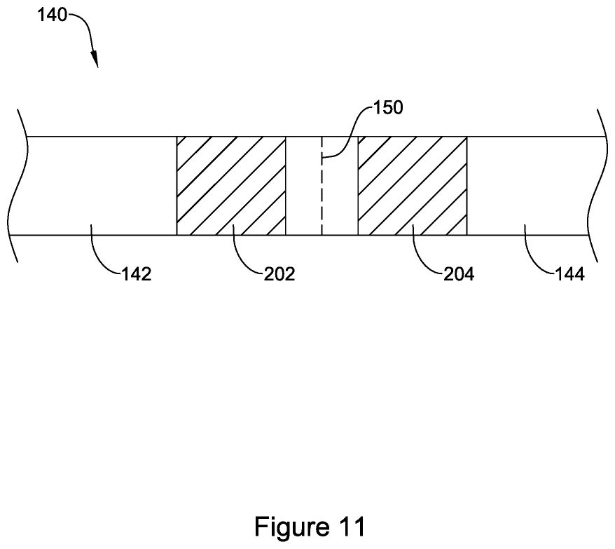

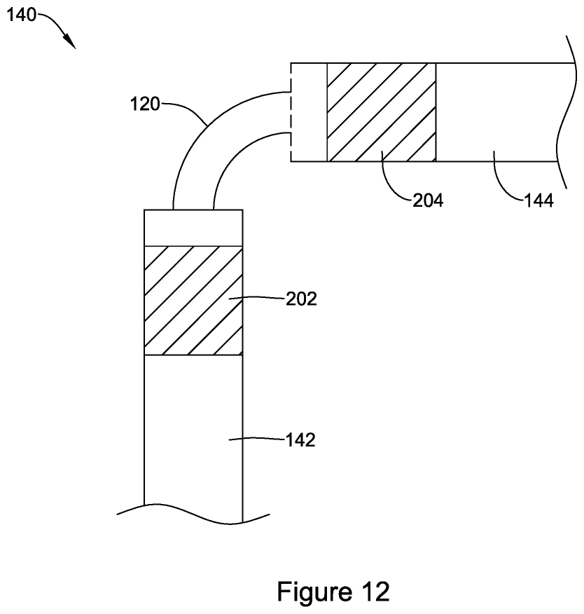

FIG. 11 and FIG. 12 together provide an example of snapping the securement member 140 (or the securement member 140a or the securement member 140b) when it is desired to release the medical device 130. The operator of the medical device system 100 may then place a first hand on the distal portion 144 of the securement member 140 and a second hand on the proximal portion 142 of the securement member 140, grasping either side of the release point 150. In some instances, the securement member 140 may include visible markers 202 and 204, positioned on either side of the release point 150 that indicate where the operator should grasp the securement member 140. The visible markers 202 and 204 may represent a color that is different from the rest of the securement member 140, for example. In some cases, the visible markers 202 and 204 may have a texture that is different from the rest of the securement member 140, providing not only a visible indication but also a tactile indication of where the operator should grasp the securement member 140 in order to snap it into two pieces in a single motion.

As an example, the operator may grasp the proximal portion 142 of the securement member 140 between their left thumb and left index finger, and may grasp the distal portion 144 of the securement member 140 between their right thumb and right index finger, the operator is able to quickly and easily snap the securement member 140 in two pieces, at the release point 150, by bending the securement member 140 at the release point 150. This is just an example, as the operator may just as easily use their right hand to grasp the proximal portion 142 of the securement member 140 and their left hand to grasp the distal portion 144 of the securement member 140, for example. FIG. 12 shows the relative position of the proximal portion 142 of the securement member 140, relative to the distal portion 144 of the securement member 140, after the operator has snapped the securement member 140 into two pieces.

Because the release wire 120 is free to move relative to the distal portion 144 of the securement member 140, and is constrained against movement relative to the proximal portion 142 of the securement member 140, the operator is able to withdraw the release wire 120 proximally by moving the proximal portion 142 of the securement member 140, which may still be in the grasp of their left hand, proximally. By virtue of moving the release wire 120 proximally, the release mechanism 170 to allowed to separate, thereby releasing the medical device 130.

The materials that can be used for the various components of the medical device system 100, the elongate shaft 110, the release wire 120, the medical device 130, the securement member 140, and/or the release mechanism 170, and/or other systems disclosed herein) and the various elements thereof disclosed herein may include those commonly associated with medical devices. For simplicity purposes, the following discussion makes reference to the medical device system 100, the elongate shaft 110, the release wire 120, the medical device 130, the securement member 140, and/or the release mechanism 170, etc. However, this is not intended to limit the devices and methods described herein, as the discussion may be applied to other elements, members, components, or devices disclosed herein, such as, but not limited to, the medical device system 100, the elongate shaft 110, the release wire 120, the medical device 130, the securement member 140, and/or the release mechanism 170, etc. and/or elements or components thereof.

In some embodiments, the medical device system 100, the elongate shaft 110, the release wire 120, the medical device 130, the securement member 140, and/or the release mechanism 170, etc., and/or components thereof (such as, but not limited to, the first portion 142, the second portion 144, the wall 146, the first portion 172, the second portion 174, etc.), may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super-elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the medical device system 100, the elongate shaft 110, the release wire 120, the medical device 130, the securement member 140, and/or the release mechanism 170, etc., and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the medical device system 100, the elongate shaft 110, the release wire 120, the medical device 130, the securement member 140, and/or the release mechanism 170, etc. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device system 100, the elongate shaft 110, the release wire 120, the medical device 130, the securement member 140, and/or the release mechanism 170, etc. to achieve the same result.

In some embodiments, a degree of Magnetic Resonance Imaging (MRI) compatibility is imparted into the medical device system 100, the elongate shaft 110, the release wire 120, the medical device 130, the securement member 140, and/or the release mechanism 170, etc. For example, the medical device system 100, the elongate shaft 110, the release wire 120, the medical device 130, the securement member 140, and/or the release mechanism 170, etc., and/or components or portions thereof, may be made of a material that does not substantially distort the image and create substantial artifacts (e.g., gaps in the image). Certain ferromagnetic materials, for example, may not be suitable because they may create artifacts in an MRI image. The medical device system 100, the elongate shaft 110, the release wire 120, the medical device 130, the securement member 140, and/or the release mechanism 170, etc., or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the medical device system 100, the elongate shaft 110, the release wire 120, the medical device 130, the securement member 140, and/or the release mechanism 170, etc., and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the medical device 130 and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A medical device system, comprising:
a medical device;
an elongate shaft having a lumen extending from a proximal end of the elongate shaft to a distal end of the elongate shaft;
a release mechanism moveable between a locked position in which the medical device is secured to the distal end of the elongate shaft and an unlocked position in which the medical device is free to move relative to the distal end of the elongate shaft;
a release wire disposed within the lumen of the elongate shaft and extendable through the release mechanism; and
a securement member including a release point formed within the securement member, the securement member including a proximal portion extending proximally from the release point and a distal portion extending distally from the release point;
wherein the release point of the securement member is configured to allow a user to grasp the proximal portion of the securement member and the distal portion of the securement member on either side of the release point and to snap the securement member into two pieces at the release point in a single motion.

2. The medical device system of claim 1, wherein the release wire extends through the securement member, with the release wire fixed relative to the proximal portion of the securement member such that once the securement member has been snapped at the release point, the proximal portion of the securement member is movable in a proximal direction in order to urge the release wire in a proximal direction.

3. The medical device system of claim 2, wherein the release wire forms a frictional fit with the proximal portion of the securement member.

4. The medical device system of claim 2, wherein the proximal portion of the securement member is crimped down onto the release wire.

5. The medical device system of claim 2, wherein the release wire is welded, soldered or adhesively secured to the proximal portion of the securement member.

6. The medical device system of claim 2, wherein the release wire is free to move relative to the distal portion of the securement member.

7. The medical device system of claim 1, wherein the release mechanism comprises:
a first release mechanism portion that is fixedly attached to the distal end of the elongate shaft; and a
second release mechanism portion that is fixedly attached to the medical device.

8. The medical device system of claim 1, wherein the securement member comprises a tubular structure made of a nickel-titanium alloy.

9. The medical device system of claim 8, wherein the tubular structure comprises:
an inner diameter (ID) that is in a range of 0.001 to 0.0049 inches; and
an outer diameter (OD) that is in a range of 0.002 to 0.0050 inches.

10. The medical device system of claim 8, wherein the release point comprises a plurality of slots extending circumferentially around the tubular structure at the release point.

11. The medical device system of claim 10, wherein the plurality of slots are radially aligned with one another, forming a plurality of beams extending circumferentially between adjacent slots.

12. The medical device system of claim 10, wherein the release point comprises a total of four radially aligned slots, with each of the slots having a length of 0.010 inches.

13. The medical device system of claim 1, wherein the proximal portion of the securement member is fixedly attached to the proximal end of the release wire and the distal portion of the securement member is fixedly attached to the proximal end of the elongate shaft.

14. The medical device system of claim 1, wherein the proximal portion of the securement member is integrally formed with the distal portion of the securement member.

15. A medical device system, comprising:
a medical device;
an elongate shaft having a lumen extending from a proximal end of the elongate shaft to a distal end of the elongate shaft;
a release mechanism moveable between a locked position in which the medical device is secured to the distal end of the elongate shaft and an unlocked position in which the medical device is free to move relative to the distal end of the elongate shaft;
a release wire disposed within the lumen of the elongate shaft and extendable through the release mechanism;
a tubular super-elastic nitinol securement member includes a release point formed within the securement member, the tubular super-elastic nitinol securement member including a proximal portion extending proximally from the release point and a distal portion extending distally from the release point;
the release point including a total of four circumferentially-arranged slots formed within the tubular super-elastic nitinol securement member, the release point being configured to allow a user to grasp the proximal portion of the securement member and the distal portion of the securement member on either side of the release point and to snap the securement member into two pieces at the release point in a single motion.

16. The medical device system of claim 15, wherein the securement member comprises:
an inner diameter (ID) that is in a range of 0.001 to 0.0049 inches; and
an outer diameter (OD) that is in a range of 0.002 to 0.0050 inches; and
the release point comprises a plurality of slots extending radially around the tubular structure.

17. A method of delivering a medical device to a treatment site, comprising:
inserting a microcatheter into a patient's anatomy and guiding a distal end of the microcatheter to a location adjacent the treatment site;
inserting a medical device disposed at a distal end of an elongate shaft into a proximal end of a lumen disposed within the microcatheter;
wherein the medical device is releasably attached to the distal end of the elongate shaft by a pull wire extending through a lumen within the elongate shaft, and wherein a securement member extends proximally from the elongate shaft, the securement member being fixedly attached to the elongate shaft and the pull wire, the securement member including a release point;
advancing the medical device through the microcatheter to the treatment site;
grasping the securement member on either side of the release point and the securement member is snapped into two pieces at the release point with a single motion, with the pull wire fixedly secured to a proximal portion of the securement member; and
translating the pull wire relative to the elongate shaft by moving the proximal portion of the securement member proximally, thereby releasing the medical device from the elongate shaft.

18. The method of claim 17, wherein the proximal portion of the securement member is fixedly attached to the pull wire and the distal portion of the securement member is fixedly attached to the elongate shaft.

19. The method of claim 18, wherein a first portion of a release mechanism is attached to the distal end of the elongate shaft and a second portion of the release mechanism is attached to a proximal end of the medical device.

20. The method of claim 19, wherein the pull wire is slidably disposed within the distal portion of the securement member, the elongate shaft, the first portion of the release mechanism, and the second portion of the release mechanism.
